(19) United States
(12) Patent Application Publication
(10) Pub. No.: US 2019/0099131 A1
(43) Pub. Date: Apr. 4, 2019

(54) TEXTILE BAND FOR TRANSMITTING ELECTRICAL SIGNAL AND SMART WEARABLE USING THE SAME

(71) Applicant: Sangmyung University Seoul Industry-Academy Cooperation Foundation, Seoul (KR)

(72) Inventors: Jung-Sim ROH, Seoul (KR); Hyo Bin IM, Seoul (KR); Hong-won PARK, Seoul (KR)

(21) Appl. No.: 16/207,681

(22) Filed: Dec. 3, 2018

Related U.S. Application Data
(63) Continuation-in-part of application No. 15/198,439, filed on Jun. 30, 2016.

(30) Foreign Application Priority Data
Mar. 21, 2016 (KR) 10-2016-0033484

(51) Int. Cl. A61B 5/00; A61B 5/11; A41D 1/00; D04B 21/20; D02G 3/38; D02G 3/44

(52) U.S. Cl. CPC A61B 5/6804; A61B 5/11; A41D 1/005; D04B 21/207; D10B 2401/061; D02G 3/441; D10B 2401/16; D10B 2331/04; D10B 2101/20; D02G 3/38

(57) ABSTRACT

Provided is a textile band for transmitting an electrical signal, which is stretched by applied tensile force to prevent damage thereto due to tensile force, and a smart wearable using the same. The textile band for transmitting the electrical signal includes yarns used as wefts, elastic threads used as warps, and a plurality of conductive yarns which are used as other warps and are disposed in a zigzag form to be stretched depending on stretching of the elastic yarns. The motion-sensing smart wearable includes an inner skin layer, to which the textile band is attached, a motion sensor module which is attached to the inner skin layer to sense a motion of the human body and transmit a sensed motion signal through the textile band, and an outer skin layer which is separably combined with the inner skin layer.

### BACKGROUND OF THE INVENTION

### 1. Field of the Invention

The present invention relates to a textile band for transmitting an electrical signal and a smart wearable using the same, and more particularly to a textile band for transmitting an electrical signal, which is stretched by applied tensile force to prevent damage thereto due to tensile force, and a smart wearable using the same.

### 2. Description of the Related Art

Recently, electrical and electronic technologies and textile technologies have converged, leading to the active development of various types of smart wearables, such as heating garments, health-monitoring garments, and electronic protectors.

Examples of the related art of smart wearables, in which electrical and electronic technologies and textile technologies are combined, include Korean Registered Utility Model No. 20-0475404 entitled “smart clothing”, Korean Registered Utility Model No. 20-0424918 entitled “external vibration adaptive smart garment”, and Korean Registered Patent No. 10-0863064 entitled “garment for measuring physiological signals and method of fabricating the same”.

A typical smart wearable includes conductive yarns in order to transmit an electrical signal. Wires are used to transmit the electrical signal in the early stages. However, a wearer may not be accepting of such wires, and garments may be poorly constructed because it is difficult to attach the wires to the garments. Therefore, currently, conductive yarns that provide wearing comfort and a fine appearance when used to stitch the garments are mainly used to transmit electrical signals in smart wearables.

The plurality of conductive yarns is weakly resistant to stretching. Therefore, the plurality of conductive yarns, which are attached to a portion of the smart wearable corresponding in position to a very active portion such as an elbow, a knee, and a shoulder, is not stretched so as to absorb tensile force, which occurs due to the wearer's motion, but receives all tensile force and is thus easily damaged.

### SUMMARY OF THE INVENTION

Accordingly, the present invention has been made keeping in mind the above problems occurring in the related art, and an object of the present invention is to provide a textile band for transmitting an electrical signal, which is stretched by applied tensile force to thus prevent the problem whereby conductive yarns, which are provided in a smart wearable to transmit electrical signals, do not absorb tensile force occurring due to the motion of the wearer but receive all tensile force to thus be damaged.

Another object of the present invention is to provide a smart wearable using the textile band for transmitting the electrical signal to sense the wearer's motion to thus correct his posture in real time.

In order to accomplish the above objects, the present invention provides a textile band for transmitting an electrical signal. The textile band includes yarns used as wefts, elastic yarns used as warps, and a plurality of conductive yarns which are used as other warps and are disposed in a zigzag form to be stretched depending on stretching of the elastic yarns.

The textile band may further include a plurality of non-conductive yarns which are used as other warps and are disposed in the zigzag form between the plurality of conductive yarns to be stretched together to thus prevent the plurality of conductive yarns from coming into contact with each other.

The plurality of conductive yarns may be coated with an insulating material.

The textile band for transmitting the electrical signal may include a transmission unit, which is fabricated using the non-conductive yarns, the elastic yarns, and the plurality of conductive yarns, and a cover which is fabricated using the non-conductive yarns and the elastic yarns and is folded to cover the transmission unit.

In order to accomplish the above objects, the present invention also provides a motion-sensing smart wearable using a textile band for transmitting an electrical signal. The motion-sensing smart wearable includes an inner skin layer, to which the textile band for transmitting the electrical signal is attached, and which comes into close contact with a human body, a motion sensor module which is attached to the inner skin layer to sense the motion of the human body and transmit a sensed motion signal through the textile band for transmitting the electrical signal, and an outer skin layer which is provided around the human body so as to cover the inner skin layer and which is separably combined with the inner skin layer.

First zippers may be provided at both sides of a front center of a zipper line in the inner skin layer, and second zippers may be provided in the front of the outer skin layer to be separably combined with the first zippers.

The motion sensor module may include a FPCB which is attached to the inner skin layer and is connected to the textile band for transmitting the electrical signal, a motion sensor which is attached to the inner skin layer to sense the motion of the human body and is connected to the FPCB to transmit the sensed motion signal through the textile band for transmitting the electrical signal, and an actuator which is attached to the inner skin layer and is connected to the FPCB to receive a control signal, which is transmitted through the textile band for transmitting the electrical signal, to thus provide a vibration.

The motion-sensing smart wearable may further include a blocking pad which is interposed between the inner skin layer and the motion sensor to prevent a motion of a skin surface from being transmitted to the motion sensor.

A textile band for transmitting an electrical signal according to the present invention includes elastic yarns, which have excellent elasticity and are used as warps, and conductive yarns, which are used as the warps and disposed in a zigzag form. Accordingly, when tensile force is applied to the textile band to elongate the textile band in the warp direction, the plurality of conductive yarns, which are disposed in the zigzag form, is also spread to be elongated, thereby preventing damage thereto due to the applied tensile force.

In addition, a motion-sensing smart wearable using the textile band for transmitting the electrical signal serves to prevent breakage of the plurality of conductive yarns of the textile band due to tensile stress caused by active motion of the wearer, and particularly senses the motion of the wearer to indicate whether his or her posture or motion is correct in real time using stimuli, thereby rapidly and precisely correcting posture during dance or exercise without help from others.

### BRIEF DESCRIPTION OF THE DRAWINGS

The patent or application file contains at least one drawing executed in color. Copies of this patent or patent application publication with color drawings will be provided by the Office upon receipt and payment of the necessary fee

The above and other objects, features and advantages of the present invention will be more clearly understood from the following detailed description taken in conjunction with the accompanying drawings, in which:

FIG. 1 is a view showing examples of a structure of a conductive yarn, which is used in the present invention, and a method of manufacturing the conductive yarn;

FIGS. 2A, 2B, and 2C are views showing examples of a structure of a textile band for transmitting an electrical signal according to the present invention and actual products of the textile band;

FIG. 3 is a picture showing an example of connection of a FPCB to the textile band for transmitting the electrical signal according to the present invention;

FIG. 4 is a view showing a structure of an upper wear article, which is used as an inner skin layer of a smart wearable according to the present invention and includes textile bands and motion sensor modules attached thereto; and

FIG. 5 is a view showing a structure of an upper wear article, which is used as an outer skin layer of the smart wearable according to the present invention.

FIG. 6 is a view showing combination of the inner skin layer and the outer skin layer of the smart wearable according to the present invention.

### DESCRIPTION OF THE PREFERRED EMBODIMENTS

Hereinafter, a detailed description will be given of a fiber band for transmitting an electrical signal and a motion-sensing smart wearable using the same according to the present invention, with reference to the appended drawings.

Prior to the specific description of the present invention, the present invention may be modified in various forms and may have a variety of embodiments, and accordingly, specific aspects (or embodiments) are illustrated in the appended drawings and are described in detail in the specification. However, the following description does not limit the present invention to the specific embodiments, and should be understood as including all variations, equivalents or substitutions within the spirit and scope of the present invention.

In the drawings, the same reference numerals, in particular the reference numerals having the same tens place and the same units place, or having the same tens place, the same units place, and the same characters denote members having the same or similar functions, and unless otherwise stated, the members designated by the reference numerals of the drawings may be understood to be based on these criteria.

Further, in the drawings, elements may be largely (or thickly) or small (or thinly) exaggerated in terms of size or thickness in consideration of the convenience of understanding, or may be simplified, but should not be construed to limit the scope of the present invention.

The terms used herein are merely intended to explain specific aspects (or embodiments) and not to limit the present invention. Unless otherwise stated, the singular expression includes a plural expression. In this application, the terms “include” or “have” are used to designate the presence of features, numbers, steps, operations, elements, parts or combinations thereof described in the specification, and should be understood as not excluding the presence or additional possibility of one or more different features, numbers, steps, operations, elements, parts or combinations thereof. Unless defined otherwise, all the terms used in this specification, including technical and scientific terms, have the same meanings as would be generally understood by those skilled in the related art. The terms defined in generally used dictionaries should be construed as having the same meanings as would be construed in the context of the related art, and unless clearly defined otherwise in this specification, should not be construed as having idealistic or overly formal meanings.

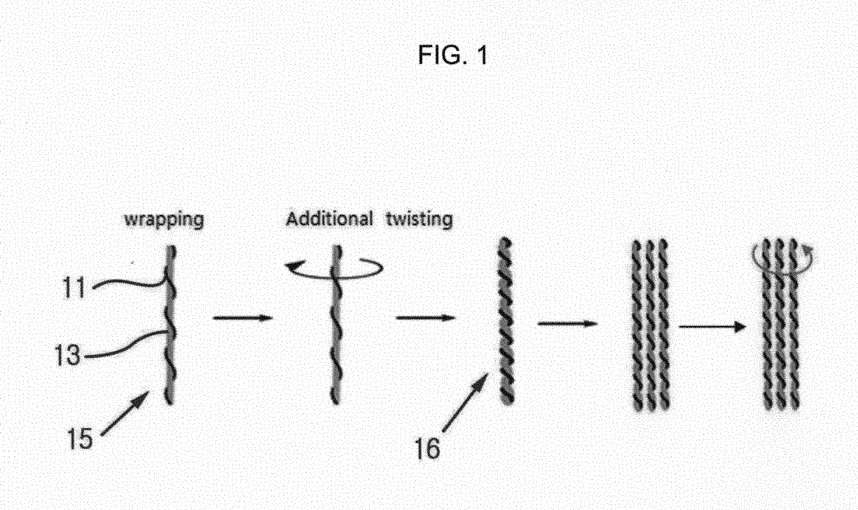

FIG. 1 shows examples of a structure of a conductive yarn, which is used in the present invention, and a method of manufacturing the conductive yarn.

The conductive yarn 10 constitutes warps of a textile band 1, and it is preferable that the conductive yarn 10 be durable so as to prevent electric disconnection from occurring during a process of knitting the textile band 1 using a warp knitting machine, and also that the conductive yarn 10 have high resistance consistency in a longitudinal direction.

For this, metal filament 11 are wrapped around polyester yarn, which are normal yarn 13, to form wrapped yarn 15, the wrapped yarn 15 are additionally twisted to form additionally twisted yarn 16, and the additionally twisted yarn 16 are combined and then twisted to thus manufacture the conductive yarn 10 according to the present invention. The manufactured conductive yarn 10 have improved durability and resistance consistency and are suitable for use as conductive yarn 10 for the smart wearable.

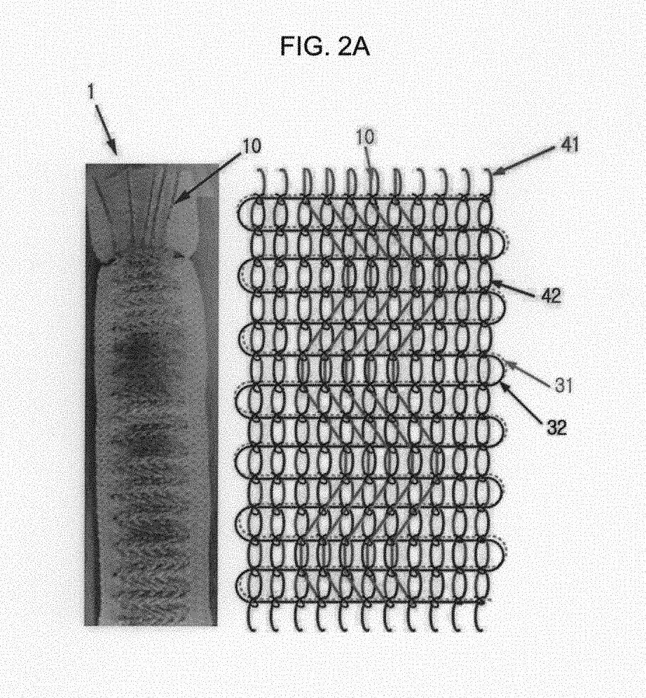

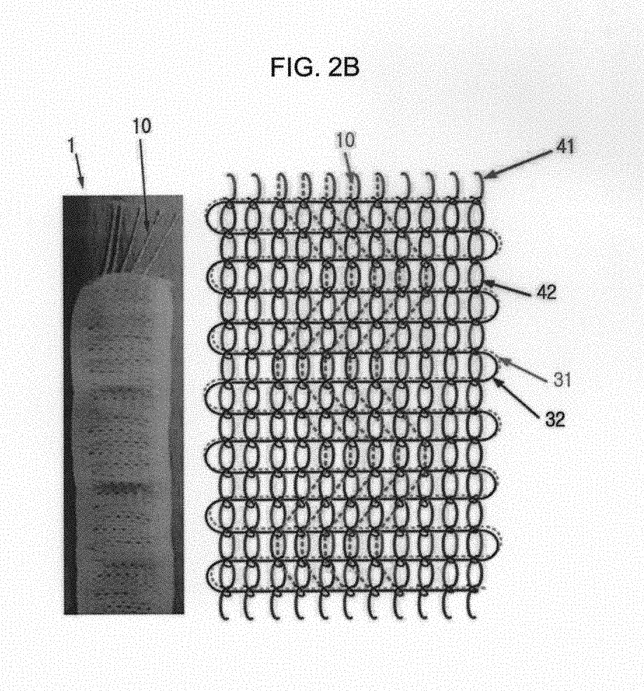

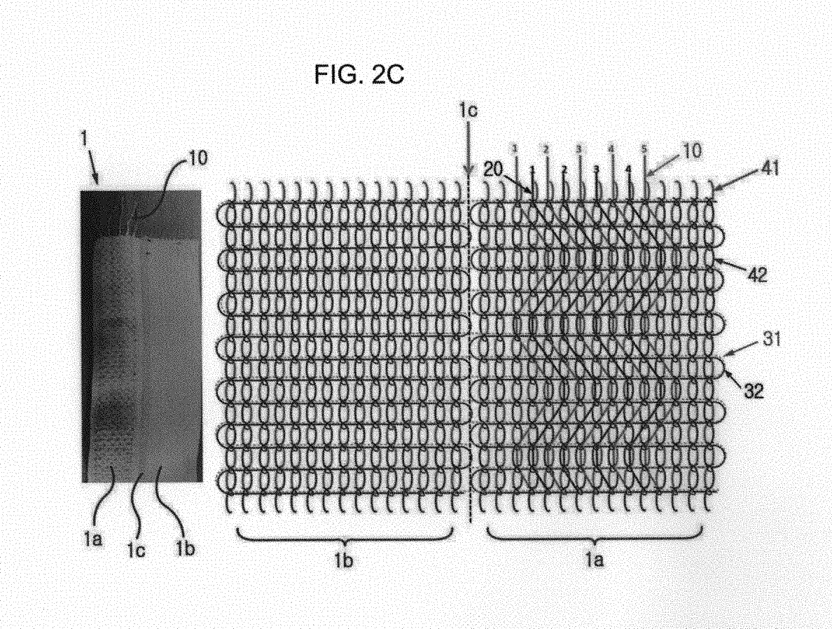

FIGS. 2A to 2C are views showing examples of the structure of the textile band 1 for transmitting the electrical signal according to various embodiments of the present invention and actual products of the textile band.

The textile band 1 includes wefts and warps. The wefts are transversely disposed and include first wefts 31 and second wefts 32 which are knitted with warps.

The warps are longitudinally disposed and include first warps 41 and second warps 42 which are knitted with the wefts.

Normal yarns, such as nylon or polyester, are used as the first wefts 31 and the second wefts 32.

Elastic yarns (for example, spandex) having high stretchability may be used as any one or both of the first warps 41 and the second warps 42 to thus elongate the first warps 41 and the second warps 42 in a warp direction when external tensile force is applied. The elastic yarns having high stretchability exhibit recovery of 99% or more after being 100% elongated.

The textile band 1 according to the present invention further includes the plurality of conductive yarns 10 as the warps (see FIG. 2A), or further includes the plurality of conductive yarns 10 and non-conductive yarns 20 as the warps (see FIG. 2B).

The plurality of conductive yarns 10 is disposed in a zigzag form in the warp direction. The plurality of conductive yarns 10 is stretched while the textile band 1 is stretched due to tensile force, thereby preventing damage thereto due to applied tensile force.

The angle and the length of the plurality of conductive yarns 10, disposed in the zigzag form, and the tension of the elastic yarns 41 during the warp knitting process may be adjusted to thereby adjust the stretchability of the textile band 1 for transmitting the electrical signal.

The metal filaments 11 of the plurality of conductive yarns 10 are the media through which the electrical signal is transmitted. When the metal filaments 11 come into contact with the metal filaments 11 of the other conductive yarns 10, electric short-circuits may occur. Therefore, a unit must be provided to prevent such electric short-circuits from occurring between the conductive threads 10.

Examples of the unit of preventing the electric short-circuits from occurring include a process of applying an insulating material on the surfaces of the metal filaments 11 of the plurality of conductive yarns 10, and a process of disposing the non-conductive yarns 20 between the plurality of conductive yarns 10 so as to prevent the plurality of conductive yarns 10 from coming into contact with each other as in FIG. 2B.

When the insulating material is applied on the metal filaments 11, there is a merit in that electric short-circuits are fundamentally prevented from occurring between the plurality of conductive yarns 10. On the other hand, in the case of a method of disposing the non-conductive yarns 20 between the transmission lines of the plurality of conductive yarns to prevent electric short-circuits from occurring between the lines, there is a merit in that the interconnection of the circuits is more easily achieved. In the former case, a process of removing the coat of the insulating material must be additionally conducted in order to connect the circuits.

The non-conductive yarns 20 are manufactured so as to have a structure which is similar to that of the plurality of conductive yarns 10 using a method similar to that of the plurality of conductive yarns 10. In other words, the two yarns are wrapped and additionally twisted, and several yarns are combined and then twisted to manufacture the non-conductive yarns 20.

As shown in FIG. 2A, the plurality of conductive yarns 10 may be exposed to a portion of the yarns. When exposed, the plurality of conductive yarns 10 may be damaged due to external impact and may come into contact with other conductive yarns 10 of the textile band 1 to thus cause short-circuits.

FIG. 2B shows the textile band 1, which includes the plurality of conductive yarns 10 covered by yarns at front and rear sides thereof, to protect the plurality of conductive yarns 10 but does not have a separate cover 1b. In the textile band 1 of FIG. 2B, the plurality of conductive yarns 10 is disposed between the first wefts 31 and the second wefts 32 so that both sides of the plurality of conductive yarns 10 are covered with the first wefts 31 and the second wefts 32 and the plurality of conductive yarns 10 is prevented from being exposed.

FIG. 2C shows the textile band 1 which has a cover 1b to prevent the conductive threads 10 from being exposed. In other words, the textile band 1 for transmitting the electrical signal according to the present invention includes a transmission unit 1a, which is fabricated using yarns 31 and 32 as the wefts and the elastic yarns 41 and the plurality of conductive yarns 10 as the warps (the yarns and the non-conductive yarns 20 may be included as the warps), and a cover 1b, which is provided beside the transmission unit la and is fabricated using the wefts and the warps without the plurality of conductive yarns 10. The cover 1b may cover the plurality of conductive yarns 10, which are exposed outside of the transmission unit 1a, to thus protect the plurality of conductive yarns 10. A crease 1c is formed between the transmission unit 1a and the cover 1b so as to make it easy to perform folding.

The metal filaments 11 of the plurality of conductive yarns 10 may be metal filaments coated with the insulating material or non-coated metal filaments.

When the metal filaments 11 coated with the insulating material are used, the insulating material may protect the metal filaments 11, and electric short-circuits due to contact between the metal filaments 11 may be prevented, and accordingly, the structure of the textile band may be simplified as in FIG. 2A. It is difficult to protect the metal filaments 11 not coated with the insulating material from an external impact when the metal filaments 11 are exposed. Therefore, the metal filaments 11 may be covered by the wefts as in FIG. 2B or may be covered by the cover 1b to be protected as in FIG. 2C, and the non-conductive yarns 20 may be disposed between the plurality of conductive yarns 10 to prevent electric short-circuits from occurring.

A PCB is connected to the textile band 1 in order to electrically connect electronic devices (for example, sensors, actuators, and coordinators), which generate electrical signals, and the textile band 1, through which the electrical signals are transmitted. Since the PCB is applied to the smart wearable, a FPCB, namely a flexible PCB, may be mainly used as the PCB.

The textile band 1 and the FPCB 131 may be connected using soldering or clamping.

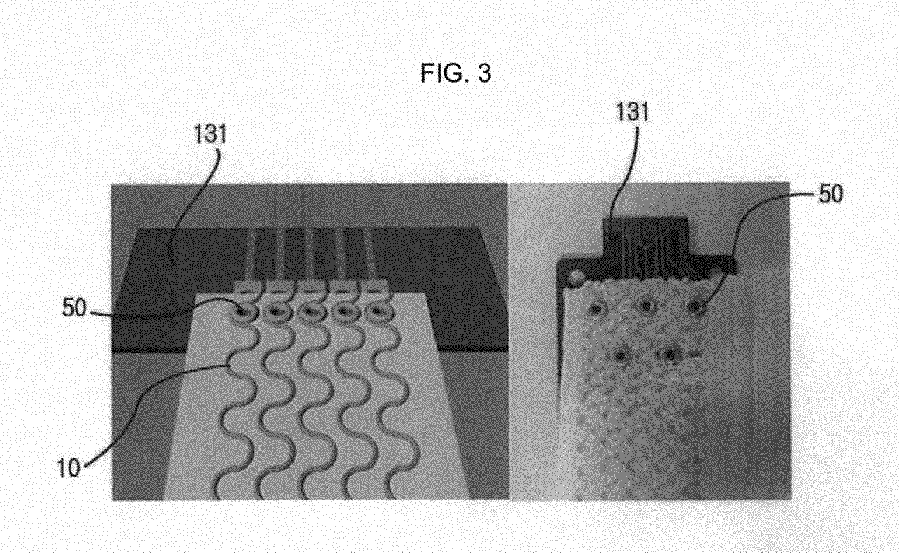

The textile band 1, which includes the plurality of conductive yarns 10 (metal filaments 11) not coated with the insulating material, is cut at a position at which it is connected with the FPCB 131. A clamping terminal 50 is provided through the connection portion of the FPCB 131 and the cut portion of the textile band 1, and both sides of the clamping terminal 50 are pressed using a press to thus bring the plurality of conductive yarns 10 into contact with the connection portion of the FPCB 131 through the clamping terminal 50, thereby electrically connecting the textile band 1 and the FPCB 131. FIG. 3 shows an example of connection of the textile band 1 and the FPCB 131 using clamping.

With regard to the plurality of conductive yarns 10 coated with the insulating material, it is difficult to bring the metal filaments 11 into direct contact with the clamping terminal 50 due to the insulating material. Accordingly, after the insulating material is removed from the plurality of conductive yarns 10 using a laser, the textile band 1 and the FPCB 131 are connected with the clamping terminal 50 by soldering or by using a conductive paste, or the textile band 1 and the FPCB 131 are soldered to be connected. In other words, the plurality of conductive yarns 10 of the textile band 1 comes into contact with the connection portion of the FPCB 131, and the molten solder paste is added to the contact portion to thus melt out the applied insulating material using the high-temperature solder paste, thereby electrically connecting the textile band 1 and the FPCB 131.

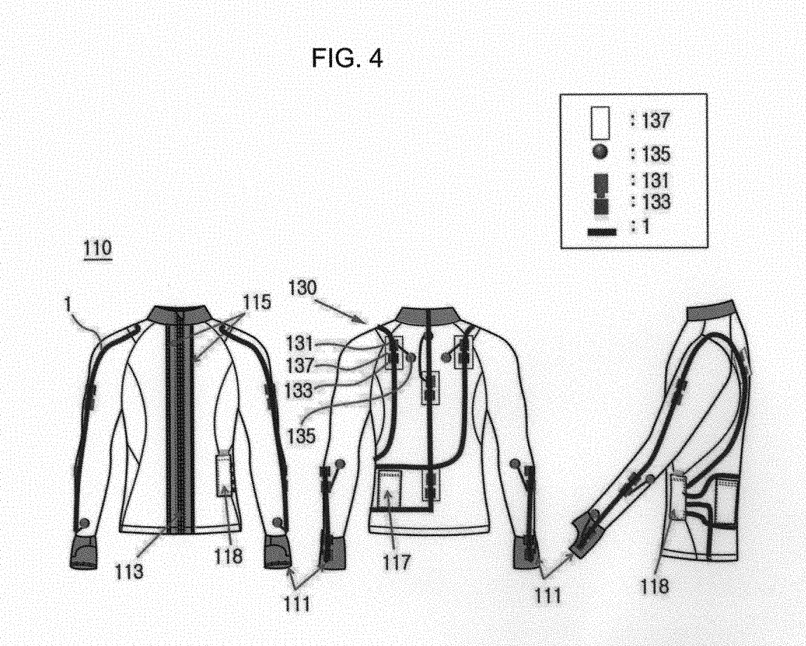

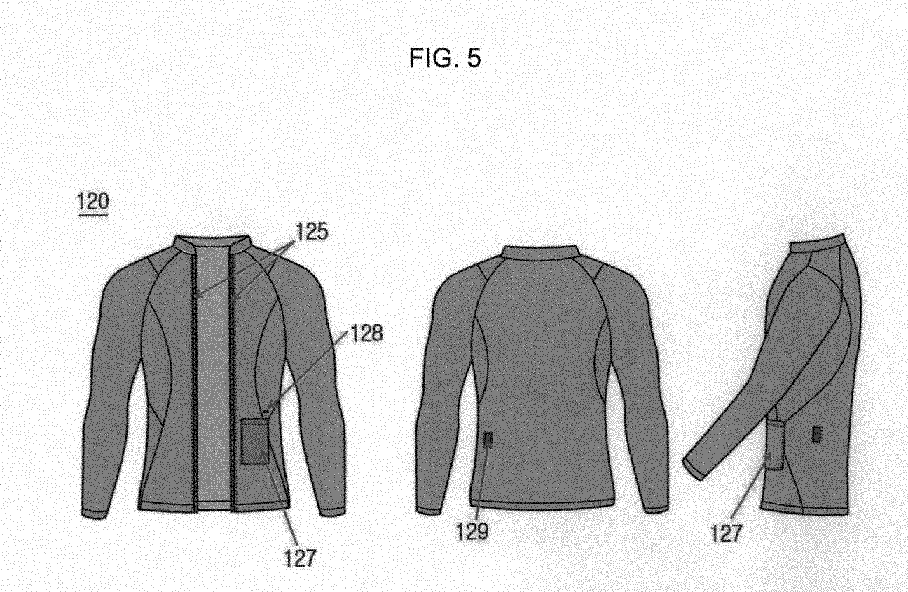

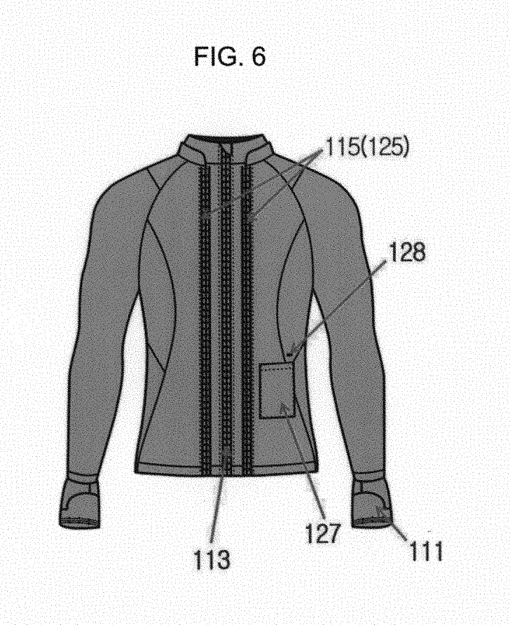

Hereinafter, a motion-sensing smart wearable using the textile band 1 for transmitting the electrical signal will be described with reference to FIGS. 4 to 6.

The smart wearable according to the present invention includes an inner skin layer 110, which comes into close contact with a human body, a motion sensor module 130, which is attached to the inner skin layer 110 and includes the textile band 1, for transmitting the electrical signal, and a motion sensor, and an outer skin layer 120 covering the inner skin layer 110. The smart wearable senses the motion of a wearer and informs the wearer in real time whether his or her posture or motion is correct, thereby rapidly and precisely correcting posture.

A stretchable material, which is manufactured by polyester, nylon, and spandex, is used as the material of the inner skin layer 110 and thus comes into close contact with the human body when the wearer wears the smart wearable. It is preferable that the motion sensor module 130 remain positioned at a specific position of the human body and that the inner skin layer 110 come into close contact with the human body in order to precisely sense the motion of the human body.

The inner skin layer 110 may include a hand back unit 111 covering the hand back of the wearer to sense the motion of the hands.

The inner skin layer 110 includes a zipper line 113, which is vertically opened and closed, at a central front side thereof so as to be easily removable from the human body. In addition, first zippers 115 are provided on both sides of the zipper line 113 so that the outer skin layer 120 is conveniently connected and separated. The first zippers 115 are connected to second zippers 125 of the outer skin layer.

The inner skin layer 110 is provided with the textile bands 1, a battery pocket 117, into which a battery is inserted to supply electric power to the motion sensor module 130, and a hub pocket 118, into which a hub is inserted. The hub is disposed at a position at which ends of the textile bands 1 are concentrated, to thus distribute transmission (transceiving) of the electrical signal through the textile bands 1.

The outer skin layer 120 is provided on the inner skin layer 110 to cover the inner skin layer 110 to thus protect the textile bands 1, the motion sensor 133, and the actuator 135 provided in the inner skin layer 110.

It is preferable that a stretchable material, which is fabricated using polyester, nylon, and spandex, be used as the material of the outer skin layer 120.

In the outer skin layer 120, the second zippers 125 are longitudinally provided at the central front side of the outer skin layer 120 so as to be connected to the first zippers 115. A pocket 127 is provided at a predetermined position on the front side, and a coordinator is inserted into the pocket 127 to gather data sensed from the motion sensor modules, compare and analyze the data, and control the driving of the actuator. A window 128 is formed over the coordinator pocket 127 to electrically connect the hub, which is inserted into the hub pocket 118 of the inner skin layer 110, and the coordinator of the coordinator pocket 127 therethrough.

Another window 129 is formed at a position on the rear side of the outer skin layer, which corresponds to the position of the coordinator pocket 127, to connect the textile bands, which are provided in the lower wear article, to the hub, which is provided in the inner skin layer 110 of the upper wear article therethrough.

The motion sensor module 130 is attached to the inner skin layer 110 and is connected to the textile bands 1 to thus sense the motion of the human body and transmit the sensed motion signal through the textile bands 1 to the coordinator.

The motion sensor module 130 includes an FPCB 131, which is connected to the textile band 1 for transmitting the electrical signal, a motion sensor 133, which senses the motion of the human body and is connected to the FPCB 131 to transmit the sensed motion signal through the textile band 1 for transmitting the electrical signal, and an actuator 135, which is connected to the FPCB 131 to receive a control signal transmitted through the textile band 1 for transmitting the electrical signal to thus provide a vibration.

The motion sensor 133 senses the attachment position, the direction, and the acceleration, and transmits the sensed motion signal through the FPCB 131 and the textile band 1 to the coordinator (not shown).

The actuator 135 is operated to provide the vibration. The actuator 135 receives a driving signal of the coordinator through the textile band 1 and the FPCB 131 to thus be driven.

Since the sensitive motion sensor 133 may also respond to a minute motion of the skin surface, it is preferable that the motion sensor 133 be provided on a blocking pad 137, and that the blocking pad 137 be attached to the inner skin layer 110 to thus prevent the minute motion of the skin surface from being transmitted to the motion sensor 133.

The FPCB 131, the motion sensor 133, and the actuator 135 constitute a set, and are attached at portions at which it is required to sense the motion of the human body.

The correction of motion using the smart wearable according to the present invention will be briefly described below.

A user wears the smart wearable and then makes a motion that the user wants to learn. For example, if the user wants to learn a golf swing motion, the user wears the smart wearable and then makes the swing motion.

When the user wearing the smart wearable makes a motion (for example, a swing motion), the motion sensors sense motion (position, direction, and acceleration) at predetermined positions on the human body.

The motion signals, which are sensed by the motion sensors 133 attached to the predetermined positions on the human body and are then transmitted, are analyzed using the coordinator to check the entire motion of the human body.

In addition, the motion data, which are stored in advance in the coordinator, are compared with the analyzed motion data to extract incorrectly moving portions and transmit the driving signal to the actuator 135 positioned at the extracted incorrectly moving portions.

The actuator 135 is driven to provide a vibration when receiving the driving signal from the coordinator to thus stimulate the corresponding portion of the human body, thereby informing the wearer which portion was moved incorrectly in real time.

The wearer checks the vibrated portion to correct his/her motion and repeats the corrected motion, thereby mastering the corrected motion.

Although the textile band for transmitting the electrical signal, which has specific shapes and structures, and the smart wearable using the same according to the present invention have been disclosed for illustrative purposes with reference to the accompanying drawings, those skilled in the art will appreciate that various modifications, additions and substitutions are possible, without departing from the scope and spirit of the invention as disclosed in the accompanying claims.

What is claimed is:

1. A motion-sensing smart wearable using a textile band for transmitting an electrical signal, the motion-sensing smart wearable comprising:
a textile band for transmitting an electrical signal, comprising yarns used as wefts, elastic yarns used as warps and a plurality of conductive yarns which are used as other warps and are disposed in a zigzag form to be stretched depending on stretching of the elastic yarns;
an inner skin layer, to which the textile band for transmitting the electrical signal is attached, and which comes into close contact with a human body;
a motion sensor module which is attached to the inner skin layer to sense a motion of the human body and transmit a sensed motion signal through the textile band for transmitting the electrical signal; and
an outer skin layer, which is provided around the human body so as to cover the inner skin layer and which is separably combined with the inner skin layer.

2. The motion-sensing smart wearable of claim 1, wherein first zippers are provided at both sides of a front center of a zipper line in the inner skin layer, and second zippers are provided in the front of the outer skin layer to be separably combined with the first zippers.

3. The motion-sensing smart wearable of claim 1, wherein the motion sensor module includes
an FPCB which is attached to the inner skin layer and connected to the textile band for transmitting the electrical signal,
a motion sensor which is attached to the inner skin layer to sense the motion of the human body and is connected to the FPCB to transmit the sensed motion signal through the textile band for transmitting the electrical signal, and
an actuator which is attached to the inner skin layer and is connected to the FPCB to receive a control signal, which is transmitted through the textile band for transmitting the electrical signal, to thus provide a vibration.

4. The motion-sensing smart wearable of claim 2, wherein the motion sensor module includes
an FPCB which is attached to the inner skin layer and connected to the textile band for transmitting the electrical signal,
a motion sensor which is attached to the inner skin layer to sense the motion of the human body and is connected to the FPCB to transmit the sensed motion signal through the textile band for transmitting the electrical signal, and
an actuator which is attached to the inner skin layer and is connected to the FPCB to receive a control signal, which is transmitted through the textile band for transmitting the electrical signal, to thus provide a vibration.

5. The motion-sensing smart wearable of claim 3, further comprising:
a blocking pad which is interposed between the inner skin layer and the motion sensor to prevent a motion of a skin surface from being transmitted to the motion sensor.

6. The motion-sensing smart wearable of claim 4, further comprising:
a blocking pad which is interposed between the inner skin layer and the motion sensor to prevent a motion of a skin surface from being transmitted to the motion sensor.
